(19) United States
(12) Patent Application Publication
(10) Pub. No.: US 2026/0225993 A1
(43) Pub. Date: Aug. 6, 2026

(54) METHOD FOR PRODUCING DIAMINE AND DICARBOXYLIC ACID, METHOD FOR RECYCLING POLYAMIDE, AND POLYAMIDE

(71) Applicants: ASAHI KASEI KABUSHIKI KAISHA, Tokyo (JP); Microwave Chemical Co., Ltd., Osaka (JP)

(72) Inventors: Yasuhisa ICHIHASHI, Tokyo (JP); Yuji WATANABE, Tokyo (JP); Ryosuke KAMOSHITA, Tokyo (JP); Satoru KAJI, Tokyo (JP); Masami YONEMURA, Tokyo (JP); Keiji KIDANI, Osaka (JP); Yuri HIRAIZUMI, Osaka (JP); Hajime KAMMIYADA, Tokyo (JP)

(73) Assignees: ASAHI KASEI KABUSHIKI KAISHA, Tokyo (JP); Microwave Chemical Co., Ltd., Osaka (JP)

(21) Appl. No.: 19/111,388

(22) PCT Filed: Sep. 24, 2024
(86) PCT No.: PCT/JP2024/033940

§ 371 Date: Mar. 13, 2025

(30) Foreign Application Priority Data
Sep. 27, 2023 (JP) 2023-165285

(51) Int. Cl. C07C 209/68; B29B 17/04; B29K 77/00; B29L 22/02; B29L 31/30; C07C 51/377; C07C 51/42; C07C 55/14; C07C 209/84; C07C 209/86; C07C 211/12; C08G 69/26; C08G 69/46; C08J 11/16

(52) U.S. Cl. CPC C07C 209/68; B29B 17/0412; C07C 51/377; C07C 51/42; C07C 209/84; C07C 209/86; C08G 69/26; C08G 69/46; C08J 11/16; B29K 2077/00; B29L 2022/027; B29L 2031/3055; C07C 55/14; C07C 211/12; C08J 2377/06

(57) ABSTRACT

A method for producing a diamine and a dicarboxylic acid, the method including a pre-treatment step of subjecting a polyamide-containing waste material to one or more selected from the group consisting of pulverization, washing, and foreign substance segregation, to obtain a crude polyamide, a depolymerization step of adding the crude polyamide to an acid-containing aqueous solution in a tightly closed reactor, raising a temperature to a target temperature of 90° C. or more and 160° C. or less, and then performing heating at or around the target temperature, to depolymerize 80% or more of an amide group in the entire polyamide contained in the crude polyamide to decompose the polyamide into a diamine and a dicarboxylic acid, a separation step of removing a component other than the diamine and a derivative of the diamine as well as the dicarboxylic acid and a derivative of the dicarboxylic acid, and a purification step.

### TECHNICAL FIELD

The present invention relates to a method for producing a diamine and a dicarboxylic acid, a method for recycling a polyamide, and a polyamide.

### BACKGROUND ART

Polyamides are excellent in mechanical properties, namely, mechanical strength, rigidity, impact resistance, and the like, and also excellent in heat resistance and chemical resistance, and therefore have been conventionally utilized in various industrial fields of clothes, industrial materials, automobiles, electrical and electronic parts, and other industrial products.

On the other hand, compatibility with the resource circulation society has also been demanded recently in the plastics industry, and establishment of recycling techniques of polyamides have also been demanded.

There are generally three kinds of recycling, including mechanical recycling, chemical recycling, and thermal recycling. Currently, in the automobile application, which accounts for much of applications of polyamides, most of polyamides in discarded automobiles are burned in thermal recycling and are not effectively utilized as resources, and it is demanded to effectively utilize such polyamides through mechanical recycling or chemical recycling.

In addition, it is demanded to subject polyamides to mechanical recycling or chemical recycling also in view of a decrease in the amount of emission of GHG (greenhouse gas).

However, a polyamide resin composition or its molded product for the automobile application includes not only a polyamide, but also, for example, an inorganic filling agent such as a glass fiber, various additives such as a thermal stabilizer, a pigment, a dye, and/or the like (see, for example, Patent Document 1), and therefore polyamides obtained from mechanical recycling have the problem of having a difficulty in keeping mechanical properties sufficient for practical use after recycling. Therefore, chemical recycling appears promising in which polyamides are depolymerized to thereby be decomposed into diamines and dicarboxylic acids as monomers and these monomers are again polymerized for recycling, and research and development thereof are advanced.

As a technique about the chemical recycling, Patent Document 2 proposes a technique for producing monomers by decomposition of a polyamide with a Lewis acid catalyst by ammonolysis. Patent Document 3 proposes a method involving separating a polyamide and a glass fiber from a molded product of a glass fiber-containing polyamide resin composition, with an aqueous phosphoric acid solution, and further decomposing the polyamide into monomers. Non-Patent Document 1 proposes depolymerization of polyamide 66 with a microwave.

### LIST OF PRIOR ART DOCUMENTS

### Patent Document

Patent Document 1: Japanese Patent No. 6839266
Patent Document 2: Japanese Patent No. 3571723
Patent Document 3: Japanese Patent Publication Laid-Open No. 2000-80199

### Non-Patent Document

Non-Patent Document 1: Urska Cesarek et al. “Chemical Recycling of Aliphatic Polyamides by Microwave-Assisted Hydrolysis for Efficient Monomer Recovery” ACS Sustainable Chem. Eng. 2020, 8, 16274-16282

### SUMMARY OF INVENTION

### Problems to be Solved by Invention

However, the method described in Patent Document 2 has the problem of low monomer yield.

Regarding the method described in Patent Document 3, a specific example thereof in which polyamide 66 is used needs as long as 200 minutes to be taken for dissolution of a polyamide and a glass fiber. Furthermore, Patent Document 3 does not provide any specific description about a depolymerization method, and moreover has the following problem: since as long as 200 minutes are taken for dissolution of a polyamide and a glass fiber, the total time including polyamide depolymerization to be performed after dissolution of the molded product of a polyamide resin composition is much longer, and thus there is lack of practicality.

The method described in Non-Patent Document 1 achieves polyamide depolymerization at a high yield in a short time, by using hydrochloric acid at a high concentration of 17% by mass or more and using a microwave to provide the state of a high temperature of 170° C. or more. Therefore, microwave irradiation with use of a glass reactor can be made at a laboratory level. However, when considering implementation at an industrial level, the material of a reaction tank is extremely limited because hydrochloric acid of a high concentration is used at a high temperature of 170° C. or more to lead to progression of corrosion of the reaction tank. For example, as described in Non-Patent Document 2 (Corrosion resistance of new metal materials, Akira Takamura, Corros. Eng., Vol. 16 (1967), No. 3, P97 to 106), even titanium, which is generally said to be high in corrosion resistance, causes the problem of corrosion in the case of hydrochloric acid at 100° C. or more and a high concentration of 5% or more.

As described above, the methods described in Patent Documents 2 and 3 disadvantageously give a low monomer yield and have problems of lack in practicality in terms of the time including that taken for monomer recovery and depolymerization. The method described in Non-Patent Document 1 has the issue about corrosion of a reaction apparatus and has the problem of being not industrially practical. In addition, if a process is performed in conditions of, for example, a high temperature and a high pressure in order to shorten the above time, the energy to perform the process is increased, and there is unfortunately no practicality also in view of a decrease in the amount of emission of greenhouse gas (GHG).

Considering the problems of the above conventional techniques, an object of the present invention is then to provide a method for producing a diamine and a dicarboxylic acid and a method for recycling a polyamide which methods can be safely carried out even under a high-pressure environment and allow for a low degree of corrosion of a reaction apparatus even when industrially carried out and which methods can achieve recovery of a diamine and a dicarboxylic acid at high yields and high purities and can use them as a polymerization feedstock to produce a polyamide comparable with a polyamide derived from a petrochemical feedstock in terms of physical properties and quality; and also to provide a polyamide obtained by the method.

### Means for Solving Problems

The present inventor has made intensive studies in order to solve the problems of the above conventional techniques, and as a result, has found that the problems can be solved by heating and decomposing a polyamide, a polyamide resin composition, and a molded product thereof in a predetermined solvent in a predetermined temperature range, separating and removing recycled feedstock-derived impurities, and foreign substances used in a recycling step or generated as by-products, and purifying a diamine and a dicarboxylic acid, thereby leading to completion of the present invention.

Specifically, the present invention is as follows.

[1]

A method for producing a diamine and a dicarboxylic acid, the method comprising
a pre-treatment step of subjecting a polyamide-containing waste material to one or more selected from the group consisting of pulverization, washing, and foreign substance segregation, to obtain a crude polyamide,
a depolymerization step of adding the crude polyamide to an acid-containing aqueous solution in a tightly closed reactor, raising a temperature to a target temperature of 90° C. or more and 160° C. or less, and then performing heating at or around the target temperature, to depolymerize 80% or more of an amide group in the entire polyamide contained in the crude polyamide to decompose the polyamide into a diamine and a dicarboxylic acid,
a separation step of removing a component other than the diamine and a derivative of the diamine as well as the dicarboxylic acid and a derivative of the dicarboxylic acid, from a reaction liquid obtained in the depolymerization step, to obtain the diamine and the derivative of the diamine as well as the dicarboxylic acid and the derivative of the dicarboxylic acid, and
a purification step of isolating and purifying each of the diamine and the dicarboxylic acid from the diamine and the derivative of the diamine as well as the dicarboxylic acid and the derivative of the dicarboxylic acid obtained in the separation step.
   [2]

The method for producing the diamine and the dicarboxylic acid according to [1], wherein, in a state where a space not filled with an aqueous solution containing the crude polyamide is present on an upper side in the reactor, and a microwave is radiated from the upper side in the reactor using a microwave transmitter through a waveguide and the space unfilled, in the depolymerization step.

[3]

The method for producing the diamine and the dicarboxylic acid according to [1] or [2], wherein a rate of temperature rise in the temperature rise is 25° C./min or less in the depolymerization step.

[4]

The method for producing the diamine and the dicarboxylic acid according to any one of [1] to [3], wherein the reactor comprises equipment which monitors a pressure in the reactor and performs evacuation when the pressure exceeds a reference pressure in the depolymerization step.

[5]

The method for producing the diamine and the dicarboxylic acid according to any one of [1] to [4], wherein the separation step comprises removing, as an insoluble fraction, a component other than the diamine and the derivative of the diamine as well as the dicarboxylic acid and the derivative of the dicarboxylic acid from the reaction liquid, in a state where a portion or an entire amount of the dicarboxylic acid and/or the derivative of the dicarboxylic acid and the diamine and the derivative of the diamine is dissolved in the reaction liquid.

[6]

The method for producing the diamine and the dicarboxylic acid according to any one of [1] to [5], wherein a pKa of the acid is 0 or less.

[7]

The method for producing the diamine and the dicarboxylic acid according to any one of [1] to [6], wherein an amount of the acid in terms of a molar ratio of an amide group of the crude polyamide and a proton of the acid satisfies the following:
Amide group of crude polyamide:Proton of acid=1:1 to 1:5.5.
   [8]

The method for producing the diamine and the dicarboxylic acid according to any one of [1] to [7], wherein the acid is hydrochloric acid.

[9]

The method for producing the diamine and the dicarboxylic acid according to [8], wherein a concentration of the hydrochloric acid in the aqueous solution is 5% by mass or more and 25% by mass or less.

[10]

The method for producing the diamine and the dicarboxylic acid according to any one of [1] to [9], wherein a main component of a polyamide in the polyamide-containing waste material is polyamide 66.

[11]

The method for producing the diamine and the dicarboxylic acid according to any one of [1] to [10], wherein the reactor used in the depolymerization step has an inner surface made of a material including a glass lining, zirconium, or tantalum.

[12]

The method for producing the diamine and the dicarboxylic acid according to any one of [1] to [11], wherein hot filtration and centrifugation are performed when the component other than the diamine and the derivative of the diamine as well as the dicarboxylic acid and the derivative of the dicarboxylic acid is removed in the separation step.

[13]

The method for producing the diamine and the dicarboxylic acid according to any one of [1] to [12], wherein in the purification step, purification of the dicarboxylic acid is performed by crystallization.

[14]

The method for producing the diamine and the dicarboxylic acid according to any one of [1] to [13], wherein in the purification step, purification of the diamine is performed by distillation.

[15]

The method for producing the diamine and the dicarboxylic acid according to any one of [2] to [14], wherein a partition window which separates the reactor and the microwave transmitter is present in the middle of the waveguide.

[16]

The method for producing the diamine and the dicarboxylic acid according to [15], wherein the partition window includes quartz glass.

[17]

The method for producing the diamine and the dicarboxylic acid according to any one of [2] to [16], wherein a frequency of the microwave in heating by the microwave is 0.8 to 6 GHz.

[18]

The method for producing the diamine and the dicarboxylic acid according to any one of [1] to [17], wherein in the purification step, a content of a Si element reaches 1 ppm by mass or more and 200 ppm by mass or less based on a total amount of the dicarboxylic acid.

[19]

A method for recycling a polyamide, the method comprising
a polymerization step of polymerizing a diamine and a dicarboxylic acid to obtain a polyamide, wherein the diamine and the dicarboxylic acid are obtained by the method for producing the diamine and the dicarboxylic acid according to any one of [1] to [18].
   [20]

A polyamide obtained by the method for recycling a polyamide according to [19], comprising 1 ppm by mass or more and 100 ppm by mass or less of a Si element based on a total amount of the polyamide.

### Advantages of Invention

According to the present invention, it is possible to provide a method for producing a diamine and a dicarboxylic acid and a method for recycling a polyamide which methods can be safely carried out even under a high-pressure environment and allow for a low degree of corrosion of a reaction apparatus even when industrially carried out and which methods can also achieve recovery of a diamine and a dicarboxylic acid at high yields and high purities and can use the resulting monomers as polymerization feedstocks to produce a polyamide comparable with a polyamide derived from a petrochemical feedstock in terms of physical properties and quality, as well as a polyamide obtained by the method.

### BRIEF DESCRIPTION OF DRAWINGS

FIG. 1 is a diagram illustrating a flowchart of a method for recycling a polyamide.

### MODE FOR CARRYING OUT INVENTION

Hereinafter, an embodiment for carrying out the present invention (hereinafter, referred to as “the present embodiment”.) is described in detail.

The following present embodiment is illustrative for describing the present invention, and is not meant to limit the present invention to the following content. The present invention can be appropriately modified and carried out within the gist thereof.

### [Method for Producing Diamine and Dicarboxylic Acid]

The method for producing a diamine and a dicarboxylic acid of the present embodiment includes
a pre-treatment step of subjecting a polyamide-containing waste material to one or more selected from the group consisting of pulverization, washing, and foreign substance segregation, to obtain a crude polyamide,
a depolymerization step of adding the crude polyamide to an acid-containing aqueous solution in a tightly closed reactor, raising the temperature to a target temperature of 90° C. or more and 160° C. or less, and then performing heating at or around the target temperature, to depolymerize 80% or more of an amide group in the entire polyamide contained in the crude polyamide to decompose the polyamide into a diamine and a dicarboxylic acid,
a separation step of removing a component other than the diamine and a derivative of the diamine as well as the dicarboxylic acid and a derivative of the dicarboxylic acid, from a reaction liquid obtained in the depolymerization step, to obtain the diamine and the derivative of the diamine as well as the dicarboxylic acid and the derivative of the dicarboxylic acid, and
a purification step of isolating and purifying each of the diamine and the dicarboxylic acid from the diamine and the derivative of the diamine as well as the dicarboxylic acid and the derivative of the dicarboxylic acid obtained in the separation step.

In the present embodiment, the diamine and the dicarboxylic acid obtained by the above are polymerized to recycle a polyamide.

The method for producing a diamine and a dicarboxylic acid and the method for recycling a polyamide of the present embodiment can be safely carried out even under a high-pressure environment and allow for a low degree of corrosion of a reaction apparatus even when industrially carried out, and can achieve recovery of a diamine and a dicarboxylic acid at high yields and high purities and provide a high-quality polyamide.

### (Pre-Treatment Step)

In the method for producing a diamine and a dicarboxylic acid of the present embodiment, a polyamide-containing waste material is subjected to one or more selected from the group consisting of pulverization, washing, and foreign substance segregation, to obtain a crude polyamide in a pre-treatment step.

### <Polyamide-Containing Waste Material>

In the method for producing a diamine and a dicarboxylic acid of the present embodiment, a polyamide-containing waste material is used. Here, the “waste material” does not mean any waste material prescribed in the law (Waste Disposal and Cleaning Act), but is defined as a collective term of a material which has been out of use as a product, a material which cannot be used as a product, and the like. The polyamide-containing waste material encompasses not only molded products and the like of a polyamide and a polyamide resin composition, but also a polyamide and a polyamide resin composition each serving as a product.

Specific examples include unnecessary odd piece and out-of-specification product from a production process of a polyamide fiber, unnecessary odd piece and out-of-specification product from a process of molding a polyamide, unnecessary odd piece and out-of-specification product from a process for weaving, sewing, or the like with a polyamide fiber, an air-bag, a carpet and an apparel product each obtained from a polyamide fiber, a molded product of a polyamide discarded after actual use, and a final product including a polyamide fiber discarded after actual use.

Hereinafter, these are collectively referred to as “polyamide-containing waste material”.

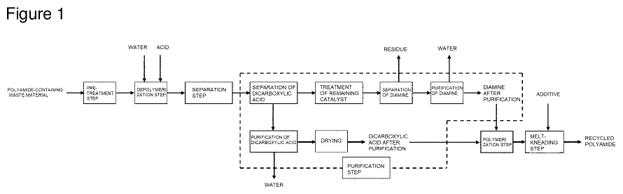

FIG. 1 illustrates a flowchart specifically representing the method for recycling a polyamide of the present embodiment.

### [Polyamide]

The “polyamide” means a polymer having an amide bond (—NHCO—) in a main chain.

The polyamide is preferably a polyamide obtained by polymerization from a diamine and a dicarboxylic acid. Examples of the polyamide include, but not limited to, polyamide 46 (polytetramethylene adipamide), polyamide 56 (polypentamethylene adipamide), polyamide 66 (polyhexamethylene adipamide), polyamide 410 (polytetramethylene sebacamide), polyamide 412 (polytetramethylene dodecamide), polyamide 610 (polyhexamethylene sebacamide), polyamide 612 (polyhexamethylene dodecamide), polyamide 1010 (polydecamethylene sebacamide), polyamide 1012 (polydecamethylene dodecamide), polyamide 6T (polyhexamethylene terephthalamide), polyamide 9T (polynonanemethylene terephthalamide), polyamide 6I (polyhexamethylene isophthalamide), and any copolymer or mixture thereof.

In particular, the polyamide preferably contains, as a main component, one or more selected from the group consisting of polyamide 66, polyamide 66/6I, polyamide 610, polyamide 612, polyamide 6I, and polyamide 6, and is more preferably polyamide 66, polyamide 66/6I, or a mixture of polyamide 66 and polyamide 6I.

Here, the “main component” means a component accounting for 50% by mass or more based on 100% by mass of the whole polymer component.

Polyamide 66 is a polyamide obtained by polycondensation of hexamethylenediamine and adipic acid, and is excellent in heat resistance, mechanical strength, and creep characteristics, and therefore suitably used as a material of a functional part for automobiles, machines, or electrical products, or as a high-strength fiber.

### [Polyamide Resin Composition]

The polyamide resin composition is a resin composition including the polyamide, and, if necessary, an inorganic filling agent such as a glass fiber, a lubricant, and/or a component as another additive, such as a thermal stabilizer, a flame retardant, a pigment, and/or a dye.

### <Inorganic Filling Agent>

The polyamide resin composition and its molded product may contain an inorganic filling agent. Thus, the polyamide resin composition and its molded product tend to have excellent mechanical strength and rigidity.

Examples of the inorganic filling agent include, but not limited to, a glass fiber, a carbon fiber, a calcium silicate fiber, potassium titanate, aluminum borate, glass flake, glass beads, talc, kaolin, mica, hydrotalcite, calcium carbonate, zinc carbonate, zinc oxide, calcium monohydrogen phosphate, wollastonite, silica, zeolite, alumina, boehmite, aluminum hydroxide, titanium oxide, silicon oxide, magnesium oxide, calcium silicate, sodium aluminosilicate, magnesium silicate, ketjen black, acetylene black, furnace black, carbon nanotube, graphite, brass, copper, silver, aluminum, nickel, iron, calcium fluoride, mica, montmorillonite, swellable fluorine mica, and apatite.

These may be used singly or in combinations of two or more thereof.

### <Lubricant>

The polyamide resin composition and its molded product may further contain a lubricant, in addition to the above polyamide resin and inorganic filling agent. Thus, the polyamide resin composition and its molded product tend to have excellent fluidity and appearance.

### <Other Additives>

The polyamide resin composition and its molded product may contain another additive, in addition to the above polyamide, inorganic filling agent, and lubricant.

Examples of the other additive include, but not limited to, an antioxidant, an ultraviolet absorber, a thermal stabilizer, a photo-degradation inhibitor, a plasticizer, a release agent, a nucleating agent, a flame retardant, a colorant, and any other thermoplastic resin.

### [Molded Product]

The molded product of the polyamide or polyamide resin composition used in the present embodiment is produced by molding by any of known various methods such as injection molding. The molded product of the present embodiment may be a fiber of the polyamide or polyamide resin composition.

Polyamides are formed into products in various forms such as fibers and films, and are used in an amount of 2000000 ton or more per year in a variety of applications such as clothes, carpets, packaging films, automobile parts, and industrial parts.

Polyamides or fiber waste materials having a higher polyamide ratio in products are suited for chemical recycling and therefore more preferred because of higher recycling efficiency. For example, base fabrics of air-bags made of polyamide 66 are optimal because they include polyamide 66 in an amount of about 90%. In addition, polyamide 66 fibers in clothing ornament such as apparel and bags, outdoor products, sportswear, or the like can also be used.

Molded products include a cable tie, which is one of representative applications of non-reinforced polyamides.

On the other hand, polyamide resin compositions of the glass fiber reinforcement grade are used for parts of automobile products in view of strength and physical properties, and the mass ratio of glass fibers in molded products thereof is most frequently 30 to 40% by mass. Therefore, polyamide components are contained at about 60 to 70% by mass, and the recycling efficiency of polyamide waste materials are lowered.

From such a viewpoint, examples of a suitable polyamide waste material for chemical recycling include a base fabric of an air-bag, a cable tie, a pile portion of a carpet, and a radiator tank, which has a glass fiber percentage of about 30% by mass, as an automobile part. Furthermore, odd pieces generated in plant during a production process are more preferable rather than used products recovered from the market because such pieces are less affected by, for example, environmental foreign substances or decomposed products due to degradation.

As illustrated in FIG. 1, the pre-treatment step of the polyamide-containing waste material is carried out in the present embodiment.

In the pre-treatment step, the polyamide-containing waste material is subjected to one or more selected from the group consisting of pulverization, washing, and foreign substance segregation, to provide a crude polyamide. Thus, a crude polyamide is obtained which is large in surface area and which allows a depolymerization step as the next step to be more efficiently performed. In this pre-treatment step, removal of metals, stone, sand, and the like as contaminants is performed. In washing, and foreign substance segregation, a specific gravity treatment by addition of washing water can be used, for example. In a case where the purity of the polyamide in the polyamide-containing waste material is high without remarkable contaminants, washing, and foreign substance segregation steps can be simplified or omitted.

A depolymerization step is performed after the pre-treatment step, whereby monomer formation can be perform at a high efficiency.

### <Depolymerization Step>

As illustrated in FIG. 1, a depolymerization step is performed after the pre-treatment step.

In the depolymerization step, the crude polyamide obtained in the pre-treatment step is added to an acid-containing aqueous solution in a tightly closed reactor, the temperature is raised to a target temperature of 90° C. or more and 160° C. or less and then heating is performed at or around the target temperature to depolymerize 80% or more of an amide group in the entire polyamide contained in the crude polyamide to thereby obtain a reaction liquid containing a diamine and a dicarboxylic acid as a result of decomposition.

Herein, the “at or around the target temperature” means a temperature range in which a depolymerization reaction stably occurs in a continuous manner.

It is necessary for cutting an amide bond of the polyamide to externally supply the energy necessary for cutting such an amide bond, and heating is needed therefor.

The method for heating is not particularly limited, and examples thereof include a method involving use of water vapor, and an electric heater. A microwave can be used to perform depolymerization at a low energy and safely carry out the depolymerization step even under a high-pressure environment.

It is necessary for promoting depolymerization to add an acid. A specific acid is described below.

Through the depolymerization step, a polyamide, a polyamide resin composition, and a molded product thereof can be decomposed into monomers at high yields, with a low energy, to carry out chemical recycling.

### [Reactor]

A tightly closed reactor is used as the reactor in the depolymerization step.

Specifically, the reactor is preferably a tightly closed pressure-resistant container because the pressure in the reactor can be increased due to vaporization of water in the reaction system, generation of outgas due to, for example, decomposition of any other organic substance than the polyamide included in the crude polyamide used as a feedstock, or generation of hydrogen gas by a reaction between a metal and an acid which can be included as foreign substances in the polyamide-containing waste material.

In a case where a microwave is used in the depolymerization step, it is preferable for efficiently irradiating the system with a microwave to not only install a microwave transmitter (magnetron) and a waveguide above the reactor, but also provide a state of the reactor in which the reactor is not fully filled with an aqueous solution containing the crude polyamide, but has an unfilled portion which is not filled with an aqueous solution containing the crude polyamide.

A microwave can be radiated from the microwave transmitter through spaces of the waveguide and the unfilled portion in this state, thereby resulting in a tendency to allow for uniform irradiation with a microwave to provide more uniform heating of the system.

In the middle of the waveguide, a partition window is preferably present which separates the reactor and the microwave transmitter and allows a microwave to penetrate though providing physical blocking.

The partition window is preferably designed so as to be able to withstand an increase in inner pressure of the reactor. Specifically, the partition window is preferably quartz glass.

The reactor preferably has equipment which always monitors the inner pressure and performs evacuation when the inner pressure exceeds a reference pressure, in view of preventing the reactor from being broken due to an abnormal increase in inner pressure by rapid gas generation.

### [Solvent]

An acid-containing aqueous solution is used in the depolymerization step. In other words, the solvent used here is water. The reason for this is as follows: a diamine, a dicarboxylic acid, derivatives thereof, and the like produced by depolymerization are soluble in water, and components insoluble in water, for example, an inorganic filler such as a glass fiber, carbon black, a pigment, and an additive are easily physically removed in removal of recovered polyamide-derived impurities in the post-process. Herein, an organic solvent such as ethylene glycol or methanol may also be used in combination as long as an appropriate process for removal of recovered polyamide-derived impurities in the post-process can be constructed. The crude polyamide, namely, the polyamide-containing waste material is not necessarily needed to be completely dissolved as a polymer in the solvent in the depolymerization step, and may be partially dissolved and decomposed into monomers in the depolymerization step. The crude polyamide is not needed to be completely dissolved as a polymer in the solvent in the depolymerization step, and therefore becoming higher viscosity in the depolymerization step is less caused advantageously so that a large amount of the crude polyamide can be added to the solvent.

### [Inorganic Salt]

An inorganic salt may be added into the aqueous solution in the depolymerization step in order to enhance the solubility of the crude polyamide, namely, the polyamide-containing waste material in water. Use of such an inorganic salt-containing aqueous solution result in a tendency to weaken a hydrogen bond between polymer chains of the polyamide and increase the solubility.

The inorganic salt is a collective term of any salt constituted from only inorganic components, and examples thereof include a metal salt which is a compound obtained by substituting a hydrogen atom of an acid with a metal ion.

Examples of the metal salt include, but not limited to, halides of calcium, zinc, lithium, chromium, iron and cobalt in view of being suitable for depolymerization, and specifically include calcium chloride, zinc chloride, zinc bromide, chromium bromide, iron bromide, lithium chloride, lithium bromide, and cobalt chloride. Preferred are calcium chloride, zinc chloride, and lithium chloride in views of availability and safety.

### [Acid]

The crude polyamide is added to an acid-containing aqueous solution in the depolymerization step.

Such an acid is considered to serve as a catalyst for hydrolysis of the polyamide.

Examples of the acid include, but not limited to, organic acids such as methanesulfonic acid, trifluoromethanesulfonic acid, p-toluenesulfonic acid, and trifluoroacetic acid; inorganic acids such as hydrochloric acid, sulfuric acid, nitric acid, and phosphoric acid; and Lewis acids such as Sc (OTf)₃, Yb (OTf)₃, Nb₂O₅, and CeO₂. These may be used singly or in combinations of a plurality thereof. An acid having a pKa of 0 or less is preferred in order to achieve a high depolymerization percentage in depolymerization.

The acid is preferably hydrochloric acid in view of the reaction efficiency, in view of decreasing impurities, and in view of reduction of a waste material which is hardly renewable.

The amount of the acid used in the depolymerization step (the number of moles of protons released) is preferably excess relative to the number of moles of amide groups in the polyamide. On the other hand, use of a large excess of the acid necessarily leads to a relative decrease in amount of the polyamide to be added in the depolymerization step, and therefore the amount of the acid is preferably not too large in view of productivity. In particular, an increase in the amount of the acid results in an increase in the amount of water in the system, and thus results in an increase in load of the water removal step in purification of a diamine in the post-process, thereby leading to an increase in cost increase and an increase in the amount of emission of GHG. In addition, use of a large excess of the acid results in production of a large amount of a salt in the neutralization step after the depolymerization step, thereby leading to an increase in load of the salt treatment process and an increase in cost. Also from this viewpoints, the amount of the acid is preferably not too large.

The molar ratio between an amide group in the crude polyamide and a proton of the acid preferably satisfies Amide group of crude polyamide:Proton of acid=1:1 to 1:5.5, more preferably 1:1 to 1:3, further preferably 1:1.15 to 1:2, from the above viewpoints.

As a result of analysis of the concentration of the acid in the aqueous solution in the depolymerization step according to the Arrhenius equation, an increase in hydrochloric acid concentration decreases the activation energy Ea to correspondingly allow a reaction to more easily progress, but simultaneously decreases water molecules to correspondingly decrease a frequency factor A, thereby causing a reaction to more hardly progress. Therefore, the hydrochloric acid concentration has an optimal region, and is preferably 5% by mass or more and preferably 25% by mass or less.

The hydrochloric acid concentration is more preferably 10% by mass to 25% by mass, further preferably 12% by mass to 22% by mass.

### [Temperature Rise]

In the depolymerization step, the crude polyamide is added to the acid-containing aqueous solution, and the temperature is raised to a target temperature of 90° C. or more and 160° C. or less.

The rate of temperature rise in the temperature rise is preferably 25° C./min or less, more preferably 20° C./min or less, further preferably 15° C./min or less in view of improving the balance between the energy applied in the temperature rise and the energy necessary for keeping a predetermined temperature after the temperature reaches such a predetermined temperature is improved and thus optimizing the equipment efficiency.

The speed of the temperature rise is preferably equal to or less than a certain value, as in the above numerical value range, also in view of preventing the reactor from being broken due to an abnormal increase in inner pressure by rapid gas generation such as generation of outgas with decomposition of any other organic substance than the polyamide included in the crude polyamide, namely, the polyamide-containing waste material or generation of hydrogen gas by a reaction between a metal and an acid which can be included as foreign substances. In the case of microwave heating, if the inner pressure is abnormally increased, heating is immediately stopped as long as supply of electricity can be stopped, and therefore a microwave is preferably used also in view of safety.

The rate of temperature rise during the heating is preferably 1° C./min or more, more preferably 2° C./min or more, further preferably 3° C./min or more in view of shortening of the time of the depolymerization step.

The target temperature is 90° C. or more and 160° C. or less, preferably 95 to 150° C., more preferably 100 to 145° C., further preferably 110 to 140° C. in view of suppression of a side reaction and in view of corrosion resistance of the reactor.

The heating method for the temperature rise preferably involves use of a microwave in views of shortening of the heating time and the effect of decreasing GHG. Any known method involving use of water vapor, an electric heater, or the like can be used. These methods may be used singly or in combinations of two or more thereof.

### [Heating]

In the depolymerization step, the temperature is raised to the target temperature as described above and then heating is performed at or around the target temperature.

The “at or around the target temperature” means that the difference between the temperature in the system during the heating and the target temperature is within 10° C., and is preferably 90° C. or more and 160° C. or less, more preferably 95 to 150° C., further preferably 100 to 145° C., still more preferably 110 to 140° C., during the heating.

As the heating method, any known method involving use of water vapor, an electric heater, or others, can be employed and a method involving use of a microwave is preferred in views of shortening of the heating time and the effect of decreasing GHG.

### [Microwave Irradiation]

The irradiation output of a microwave during the heating may be appropriately selected, and may be an output capable of providing heating to the above temperature. The upper limit of the irradiation output is not particularly limited.

The frequency of a microwave is not particularly limited, and is, for example, preferably 0.8 to 6 GHz. The frequency is more preferably 0.8 to 2.5 GHz, further preferably 0.8 to 1.5 GHz, still more preferably 0.8 to 1 GHz, still further preferably 0.9 to 0.95 GHz in view of allowing a microwave to more easily reach the system in the depolymerization step.

### [Corrosion Resistance]

The reactor used in the depolymerization step is preferably one which is difficult to corrode and which has industrial practicality. Specifically, a reactor is preferably used which has an inner surface made of a material such as a glass lining, zirconium, or tantalum. Such corrosion resistance can be determined from, for example, the data described in “Properties of Tantalum for Applications in the Chemical Process Industry: F. J. Hunkeler (USA) ASTM STP849, 1984, P28-49”.

### [Depolymerization Percentage of Polyamide in Depolymerization Step]

In the depolymerization step, 80% or more of an amide group in the entire polyamide included in the crude polyamide is depolymerized, in view of achieving a high recovery percentage in the method for producing a diamine and a dicarboxylic acid of the present embodiment.

On the other hand, a dicarboxylic acid, a diamine, and derivatives thereof, as decomposed products, account for 80% by mass or more of the entire polyamide included in the crude polyamide, from the standpoint for recovery of such decomposed products.

The residual rate of the polyamide, in terms of an amide group in the entire polyamide included in the crude polyamide, is preferably 10% or less, more preferably 7% or less, further preferably 5% or less, still further preferably 1% or less.

The depolymerization percentage, in terms of an amide group decomposed in the entire polyamide included in the crude polyamide, is preferably 85% or more, more preferably 90% or more, further preferably 95% or more of an amide group.

The depolymerization percentage of the polyamide, the amount of the decomposed products and the residual rate of the polyamide can be controlled within the above numerical value ranges by adjusting the acid concentration, and the reaction time and temperature.

### <Separation Step>

As illustrated in FIG. 1, the separation step is performed after the above depolymerization step in the present embodiment.

In the separation step, in a state where a portion or the entire amount of the dicarboxylic acid and the derivative of the dicarboxylic acid is dissolved in the reaction liquid, a component other than the diamine and the derivative of the diamine as well as the dicarboxylic acid and the derivative of the dicarboxylic acid is removed as an insoluble fraction, from the reaction liquid obtained in the depolymerization step, to obtain the diamine and the derivative of the diamine as well as the dicarboxylic acid and the derivative of the dicarboxylic acid.

The polyamide-containing waste material, and the crude polyamide obtained in the pre-treatment step may each include a glass fiber, a carbon fiber, any other inorganic filling agent, various coating agents each having a water-repelling function, and/or contamination-derived impurities such as sand. These remain as solid contents insoluble in the reaction liquid after the depolymerization step, and thus are mostly removed in the separation step. The separation method is not particularly limited, and examples thereof include precipitation separation, centrifugation, and filtration of the insoluble fraction.

A specific method for separating the component other than the diamine and the derivative of the diamine as well as the dicarboxylic acid and the derivative of the dicarboxylic acid can be, for example, any known method such as filtration with a filter, or a method involving use of an ion-exchange membrane, an ion-exchange resin, or the like, depending on an objective substance to be removed. The polyamide-containing waste material generally includes a metal, glass, a glass fiber, sand, and the like and therefore such foreign substances are preferably removed by hot filtration or the like while the temperature of the reaction liquid is still high immediately after the depolymerization step, in view of an increase in process efficiency and saving energy. The temperature of such hot filtration is preferably a temperature at which none of the diamine and the derivative of the diamine as well as the dicarboxylic acid and the derivative of the dicarboxylic acid are precipitated. Therefore, the temperature is preferably 55° C. or more, more preferably 60° C. or more, further preferably 65° C. or more.

In the present embodiment, in order to enhance the recovery percentages of the diamine and the dicarboxylic acid, the insoluble fraction is preferably separated in the separation step in a state where a portion or the entire amount of the dicarboxylic acid and the derivative of the dicarboxylic acid and a portion or the entire amount of the diamine and the derivative of the diamine are dissolved in the reaction liquid obtained in the above depolymerization step, for example, in a temperature range where these are dissolved therein, and the insoluble fraction is more preferably separated in the separation step in a state where the entire amounts thereof are dissolved.

Herein, the entire amount of the insoluble fraction is not necessarily needed to be removed in the separation step, and a solid content slightly remaining can also be removed again in the purification step described below, which is for isolation and purification of the diamine and the dicarboxylic acid.

In the present embodiment, a dicarboxylic acid precipitated from the reaction liquid and at the same time an insoluble solid content are preferably removed in the purification step described below. In this case, such an insoluble solid content can also be removed in the post-process, by fully dissolving selectively such a dicarboxylic acid in a solvent, from a mixture of such a dicarboxylic acid and such an insoluble solid content, and separating only the insoluble fraction from the resulting solution.

### <Purification Step>

As illustrated in FIG. 1, a purification step is performed after the separation step in the present embodiment.

In the purification step, each of the diamine and the dicarboxylic acid is isolated and purified from the diamine and the derivative of the diamine as well as the dicarboxylic acid and the derivative of the dicarboxylic acid obtained in the separation step.

Any known method can be used in the purification step of isolating and purifying each of the diamine and the dicarboxylic acid, and is not particularly limited, and examples thereof include the following methods.

### [Isolation-Purification Step of Diamine]

In the isolation-purification method of the diamine in the purification step, a diamine salt of the diamine with a dicarboxylic acid (for example, adipic acid) or an acid (for example, hydrochloric acid) is dissolved in the liquid after the separation step. Examples of the method for separating the diamine from a diamine salt-containing solution include a neutralization treatment. The diamine can be released from the diamine salt by adjusting the pH in the range of 7 to 14. The alkali used in the neutralization treatment may be any alkali as long as it has a higher pKa than the diamine to be separated as an objective substance, and the alkali industrially publicly used is, for example, sodium hydroxide, calcium hydroxide, or potassium hydroxide.

Examples of the method for purifying the diamine released to a purity applicable for polyamide polymerization include purification by distillation. Such purification by distillation can also allow a residue included in the crude polyamide to be removed. In a case where a compound having the carboxylic acid is included in the reaction liquid after the separation step, the diamine and the carboxylic acid are polymerized during heating in distillation, leading to a reduction in yield of the diamine and scaling to an apparatus. Therefore, the carboxylic acid is preferably removed before such distillation, by a method such as crystallization, physical adsorption by activated carbon or an ion-exchange resin, or membrane separation.

Examples of still other diamine purification methods include a method by extraction with a solvent forming an organic phase, and a method by membrane separation.

### [Isolation-Purification Step of Dicarboxylic Acid]

In the isolation-purification method of the dicarboxylic acid in the purification step, purification by crystallization is preferably performed after the separation step.

Examples of such purification by crystallization include a method including precipitating the dicarboxylic acid from the reaction liquid obtained in the separation step to crystallize the dicarboxylic acid by recrystallization, performing solid-liquid separation to obtain a crude dicarboxylic acid crystal, furthermore adding and dissolving the obtained crude dicarboxylic acid crystal in pure water, and then subjecting the resultant to crystallization and solid-liquid separation, and drying to thereby obtaining a purified dicarboxylic acid.

During crystallization, the solution may be stirred and/or heated to dissolve the crude dicarboxylic acid crystal, and/or may be aged for an appropriate time to provide crystal growth. Proper conditions may be selected as the drying conditions as long as a temperature equal to or less than the melting point of the dicarboxylic acid is adopted.

The dicarboxylic acid obtained in the separation step, which is different from a dicarboxylic acid made by a usual production method, is in the form in which any additive, and pigment-derived metal compound and organic compound remain as impurities. These impurities cause coloration of the dicarboxylic acid and/or serve as a polymerization inhibitor during re-polymerization to a polyamide, and therefore are preferably removed by crystallization in the isolation-purification step. Examples of a suitable method include application of a method involving subjecting the dicarboxylic acid obtained by crystallization to washing with an inorganic acid such as nitric acid, sulfuric acid, or hydrochloric acid, physical adsorption with an ion-exchange resin or activated carbon, membrane separation, or the like.

The dicarboxylic acid thus purified can be recovered as a crystalline dicarboxylic acid by drying remaining water, or can be mixed with the diamine without drying and then utilized as a dicarboxylic acid-diamine salt.

In the present embodiment, preferably, first the dicarboxylic acid is separated from the reaction liquid obtained in the separation step, thereafter a treatment of the remaining catalyst is performed, the diamine is then separated, and each of the dicarboxylic acid and the diamine is purified, as illustrated in FIG. 1.

### [Content of Si Element]

In the purification step, the content of a Si element is preferably 1 ppm by mass or more based on the total amount of the dicarboxylic acid in view of a proper viscosity of the polyamide and thus stabilized polymerization when a polyamide is produced by polymerization of the dicarboxylic acid and the diamine, and in view of improved handleability of the resulting polyamide. The content is preferably 200 ppm by mass or less in view of preventing inhibition of polymerization in production of a polyamide by polymerization of the dicarboxylic acid and the diamine.

The content of a Si element is more preferably 1 to 150 ppm by mass, further preferably 1 to 120 ppm by mass, still more preferably 1 to 100 ppm by mass.

The Si element is derived from a waste polyamide and the content thereof can be controlled within the above numerical value range by adjusting the purification step.

### [Method for Recycling Polyamide]

The method for recycling a polyamide of the present embodiment includes the polymerization step of polymerizing a diamine and a dicarboxylic acid obtained by the method for producing a diamine and a dicarboxylic acid of the present embodiment to obtain a polyamide, as illustrated in FIG. 1. Thus, a polyamide can be recycled. A known method can be used in the polymerization step, and examples thereof include the following method, but not limited thereto.

### (Polymerization Step)

In the polymerization step, a method commonly and frequently used is a method in which an aqueous solution of a dicarboxylic acid-diamine salt or a mixture of the dicarboxylic acid and the diamine, or a suspension thereof in water is heated and subjected to polymerization with a melted state being kept (hereinafter, also referred to as “hot melt polymerization method”.). However, the method for polymerization is not limited thereto, and polymerization can be made by a known method such as a solid phase polymerization method or a solution method.

Examples of a specific method of producing a polyamide from the diamine and the dicarboxylic acid after purification include various methods exemplified below.
(1) A method in which an aqueous solution of a dicarboxylic acid-diamine salt or a mixture of the dicarboxylic acid and the diamine, or a suspension thereof in water is heated and subjected to polymerization with a melted state being kept (hereinafter, also referred to as “hot melt polymerization method”.).
(2) A method in which, while the polyamide obtained in the hot melt polymerization method is kept in a solid state at a temperature equal to or less than the melting point of the polyamide, the degree of polymerization is increased (hereinafter, also referred to as “hot melt polymerization/solid phase polymerization method”.).
(3) A method in which a dicarboxylic acid-diamine salt or a mixture of the dicarboxylic acid and the diamine is polymerized with a solid state being kept (hereinafter, also referred to as “solid phase polymerization method”.).
(4) A method in which polymerization is made by using a dicarboxylic acid halide component equivalent to the dicarboxylic acid and a diamine component (hereinafter, also referred to as “solution method”.).

In particular, a production method including the hot melt polymerization method is preferred, and a melted state is preferably retained until the termination of polymerization, in production of a polyamide by the hot melt polymerization method. It is necessary for retaining a melted state to perform production in polymerization conditions suitable for the compositional ratio of the polyamide. For example, the following method can be used: while the polymerization pressure in the hot melt polymerization method is controlled to 14 to 25 kg/cm² (gauge pressure) and heating is continued, pressure drop is made over 30 minutes or more until the pressure in the tank reaches the atmospheric pressure (gauge pressure 0 kg/cm²).

The manner for forming a polyamide by polymerization is not particularly limited, and may be either a batch type or a continuous type.

The polymerization apparatus used in production of a polyamide is not particularly limited, any known apparatus can be used, and examples thereof include an autoclave-type reactor, a tumbler-type reactor, and an extruder-type reactor such as a kneader.

Hereinafter, a method for producing a polyamide by a batch-type hot melt polymerization method is specifically shown as the production method of a polyamide, but the production method of a polyamide is not limited thereto.

First, for example, an aqueous solution containing about 40 to 60% by mass of feedstock components (dicarboxylic acid, diamine, and, if necessary, lactam and/or aminocarboxylic acid) of a polyamide is concentrated to about 65 to 90% by mass in a concentration tank operated at a temperature of 110 to 180° C. and a pressure of about 0.035 to 0.6 MPa (gauge pressure), thereby obtaining a concentrated solution.

Next, the concentrated solution obtained is transferred to an autoclave, and heating is continued until the pressure in the autoclave reaches about 1.2 to 2.2 MPa (gauge pressure).

Thereafter, the pressure is kept at about 1.2 to 2.2 MPa (gauge pressure) with water and/or a gas component being drained in the autoclave, and once the temperature reaches about 220 to 260° C., the pressure is dropped to the atmospheric pressure (gauge pressure 0 MPa).

The pressure in the autoclave can be dropped to the atmospheric pressure, and then, if necessary, reduced, thereby effectively removing water as a by-product.

Thereafter, the inside of the autoclave is pressurized with an inert gas such as nitrogen, and a polyamide melt is extruded as a strand from the autoclave. The strand extruded is cooled and cut to obtain a polyamide pellet.

The content of a Si element in a polyamide obtained by the method for recycling a polyamide is preferably 1 ppm by mass or more based on the total amount of the polyamide in view of a not too high viscosity and therefore improved handleability of a melt-kneaded product when an additive is added to perform melt-kneading. The content is also preferably 100 ppm by mass or less in view that a reduction in molecular weight of the polyamide is controlled.

The content is more preferably 1 to 50 ppm by mass, further preferably 1 to 30 ppm by mass.

The polyamide obtained in the polymerization step may be compounded with various additives depending on desired physical properties, as illustrated in FIG. 1. The polyamide is melt-kneaded to obtain a recycled polyamide as a final object.

### EXAMPLES

Hereinafter, the present invention is further specifically described with reference to specific Examples and Comparative Examples, but the present invention is not limited by the following Examples and Comparative Examples at all.

The following feedstocks and others were used to obtain purified monomers and polymers in the following steps in Examples and Comparative Examples, and evaluation was performed by the following methods.

### [Polyamide, Polyamide Resin Composition, and Polyamide-Containing Waste Material Used in Chemical Recycling]

### (Polyamide)

A: polyamide 66 (manufactured by Asahi Kasei Corporation, Model number: Leona 1300)

### (Polyamide Resin Composition)

B: polyamide 66 resin composition (manufactured by Asahi Kasei Corporation, Model number: Leona 14G33, glass fiber ratio 33%)

In Examples and Comparative Examples described below, such polyamide and polyamide resin composition were also used as “polyamide-containing waste material” and “crude polyamide” in addition to the following waste material, mainly in light of utilization of odd pieces and stock recycling.

### (Waste Material of Molded Product of Polyamide Resin Composition)

A cap of a radiator tank was recovered from an automobile discarded.

The cap was stamped with >PA66+GF30<, in which polyamide 66 was a main component and 30% by mass of a glass fiber was included.

### (Fabric-Like Waste Material of Polyamide Fiber)

An air-bag was recovered from an automobile discarded.

It was stamped with PA66, in which polyamide 66 was a main component.

Both a colored air-bag (coated with silicon) and a blank air-bag (not coated with silicon) were used.

### [Pre-Treatment Step of Pulverizing Polyamide-Containing Waste Material to Produce Crude Polyamide]

### (Pulverization of Molded Product of Polyamide Resin Composition)

A molded product of the polyamide resin composition was pulverized with a pulverizer “PFS-40” manufactured by Nihon-Cim. After pulverization, an irregular resin piece having a length of 3 to 6 mm was obtained.

### (Pulverization of Fabric-Like Waste Material (Air-Bag) of Polyamide Fiber)

An air-bag odd piece and a discarded air-bag were each shredded to a proper size.

Although the shredding dimension is preferably having a length of one side of about 1 cm to 10 cm, the dimension is varied depending the equipment used and the throughput, and therefore the shredding dimensions of the air-bag odd piece and the waste air-bag were not particularly restricted.

### (Handling of Polyamide 66 and Polyamide 66 Resin Composition)

The polyamide 66 (manufactured by Asahi Kasei Corporation, Model number: Leona 1300) and the polyamide 66 resin composition (manufactured by Asahi Kasei Corporation, Model number: Leona 14G33, glass fiber ratio 33%) were each directly used in the form of a pellet, as a crude polyamide.

### [Solvent Used in Depolymerization Step]

### (Acid)

The following inorganic acids and organic acids were each used as the acid.

Hydrochloric acid (special grade-purity 35 to 37%) manufactured by Kanto Kagaku

Sulfuric acid (special grade-purity >96%) manufactured by Kanto Kagaku

Phosphoric acid (special grade-purity 85%) manufactured by Hayashi Pure Chemical Ind., Ltd.

Methanesulfonic acid manufactured by TCI Corporation

p-Toluenesulfonic acid monohydrate manufactured by TCI Corporation

### [Alkali Used in Purification Step of Isolating-Purifying Diamine]

### (Alkali)

The following sodium hydroxide was used as the alkali for neutralization of the acid in the purification step.

Sodium hydroxide manufactured by TCI Corporation

### [Production of Sample Liquid Used in Depolymerization Step]

The inorganic acid or organic acid, water, the polyamide, the polyamide resin composition, and the crude polyamide were weighed in a 20-mL glass pressure-resistant test tube (Reaction Vial G30 manufactured by Anton Paar GmbH), according to the following Table 1 to Table 3.

In the case of the polyamide resin composition, the amount of resin components, except for the mass of the inorganic filler, was adopted as the amount of polyamide, and the amount of polyamide was adjusted so as to be the same as that in other system.

Table 1 to Table 3 show the compositional ratio of each of Sample Liquids 1 to 18 to be used in Examples and Comparative Examples.

TABLE 1

| | | | Sample | Sample | Sample | Sample | Sample | Sample |
|---|---|---|---|---|---|---|---|---|
| | | Note | liquid 1 | liquid 2 | liquid 3 | liquid 4 | liquid 5 | liquid 6 |
| Inorganic | Hydrochloric acid (g) | pKA = −8.0 | 4.8 | 1.8 | 1.4 | 1.2 | — | — |
| acid | Sulfuric acid (g) | pKA = −3.0 | — | — | — | — | 3.5 | — |
| | Phosphoric acid (g) | pKA = 2.12 | — | — | — | — | — | 2.3 |
| | Adipic acid (g) | pKA = 4.42 | — | — | — | — | — | — |
| Organic | Methanesulfonic acid (g) | pKA = −2.6 | — | — | — | — | — | — |
| acid | p-Toluenesulfonic acid (g) | pKA = −2.8 | — | — | — | — | — | — |
| Water (g) | | | 8.9 | 8.9 | 8.9 | 8.9 | 8.2 | 8.8 |
| Acid concentration (% by mass) | | Acid/(Acid + | 35 | 17 | 14 | 12 | 30 | 21 |
| | | Water | | | | | | |
| Polyamide | Leona 1300 pellet (g) | Polyamide ratio | 1.12 | 1.12 | 1.12 | 1.12 | 1.12 | 1.12 |
| | | 100% | | | | | | |
| | Leona 14G33 pellet (g) | Polyamide ratio | — | — | — | — | — | — |
| | | 67% | | | | | | |
| | Pulverized product of radiator tank | Polyamide ratio | — | — | — | — | — | — |
| | recovered from market (g) | 70% | | | | | | |
| | Cut product of air-bag | Polyamide ratio | — | — | — | — | — | — |
| | (coated with silicon) (g) | 95% | | | | | | |
| | Cut product of air-bag | Polyamide ratio | — | — | — | — | — | — |
| | (not coated with silicon) (g) | 100% | | | | | | |

TABLE 2

| | | | Sample | Sample | Sample | Sample | Sample | Sample |
|---|---|---|---|---|---|---|---|---|
| | | Note | liquid 7 | liquid 8 | liquid 9 | liquid 10 | liquid 11 | liquid 12 |
| Inorganic | Hydrochloric acid (g) | pKA = −8.08.0 | — | — | — | 1.8 | 1.8 | 1.8 |
| acid | Sulfuric acid (g) | pKA = −8.03.0 | — | — | — | — | — | — |
| | Phosphoric acid (g) | pKA = 2.12 | — | — | — | — | — | — |
| | Adipic acid (g) | pKA = 4.42 | 3.0 | — | — | — | — | — |
| Organic | Methanesulfonic acid (g) | pKA = −2.6 | — | 2.5 | — | — | — | — |
| acid | p-Toluenesulfonic acid (g) | pKA = −2.8 | — | — | 4.7 | — | — | — |
| Water (g) | | | 7.0 | 8.3 | 8.3 | 8.9 | 8.9 | 8.9 |
| Acid concentration (% by mass) | | Acid/(Acid + | 30 | 23 | 36 | 17 | 17 | 17 |
| | | Water) | | | | | | |
| Polyamide | Leona 1300 pellet (g) | Polyamide ratio | 1.12 | 1.12 | 1.12 | — | — | — |
| | | 100% | | | | | | |
| | Leona 14G33 pellet (g) | Polyamide ratio | — | — | — | 1.67 | — | — |
| | | 67% | | | | | | |
| | Pulverized product of radiator tank | Polyamide ratio | — | — | — | — | 1.6 | — |
| | recovered from market (g) | 70% | | | | | | |
| | Cut product of air-bag | Polyamide ratio | — | — | — | — | — | 1.18 |
| | (coated with silicon) (g) | 95% | | | | | | |
| | Cut product of air-bag | Polyamide ratio | — | — | — | — | — | — |
| | (not coated with silicon) (g) | 100% | | | | | | |

TABLE 3

| | | | Sample | Sample | Sample | Sample | Sample | Sample |
|---|---|---|---|---|---|---|---|---|
| | | Note | liquid 13 | liquid 14 | liquid 15 | liquid 16 | liquid 17 | liquid 18 |
| Inorganic | Hydrochloric acid (g) | pKA = −8.0 | 1.8 | 1.8 | 1.8 | 1.8 | 1.8 | 1.8 |
| acic | Sulfuric acid (g) | pKA = −3.0 | — | — | — | — | — | — |
| | Phosphoric acid (g) | pKA = 2.12 | — | — | — | — | — | — |
| | Adipic acid (g) | pKA = 4.42 | — | — | — | — | — | — |
| Organic | Methanesulfonic acid (g) | pKA = −2.6 | — | — | — | — | — | — |
| acid | p-Toluenesulfonic acid (g) | pKA = −2.8 | — | — | — | — | — | — |
| Water (g) | | | 8.9 | 8.9 | 8.9 | 8.9 | 8.9 | 8.9 |
| Acid concentration (% by mass) | | Acid/(Acid + | 17 | 17 | 17 | 17 | 17 | 17 |
| | | Water) | | | | | | |
| Polyamide | Leona 1300 pellet (g) | Polyamide ratio | — | 2.5 | 3 | 3.5 | 4 | 4.5 |
| | | 100% | | | | | | |
| | Leona 14G33 pellet (g) | Polyamide ratio | — | — | — | — | — | — |
| | | 67% | | | | | | |
| | Pulverized product of radiator tank | Polyamide ratio | — | — | — | — | — | — |
| | recovered from market (g) | 70% | | | | | | |
| | Cut product of air-bag | Polyamide ratio | — | — | — | — | — | — |
| | (coated with silicon) (g) | 95% | | | | | | |
| | Cut product of air-bag | Polyamide ratio | 1.12 | — | — | — | — | — |
| | (not coated with silicon) (g) | 100% | | | | | | |

### [Examples 1 to 37] [Comparative Examples 1 to 16]

Each of Sample Liquid 1 to 18 shown in Table 1 to Table 3 was used to perform the depolymerization step in conditions of Tables 4 to Table 8, and Table 10.

Table 9 shows the analysis results of hexamethylenediamine after purification and the analysis results of polyamide 66 after polymerization.

### [Depolymerization Step]

### <Microwave Heating>

A pressure-resistant test tube was charged with a stirrer and predetermined amounts of the polyamide, the polyamide resin composition and the crude polyamide, and 37% hydrochloric acid and distilled water were added thereto so that a desired percentage (% by mass) was achieved. The resulting mixture was stirred with a microwave synthesis reactor (Monowave 450 manufactured by Anton Paar GmbH) at a predetermined temperature for a desired time. The temperature rise time was fixed to 16 minutes in the temperature rise process to a predetermined temperature, and the number of rotations of the stirrer was set to 600 rpm.

After depolymerization was performed at a predetermined temperature for a desired time, the resultant was naturally cooled in an apparatus and, when the temperature was dropped to 70° C., the pressure-resistant test tube was taken out.

For scaling up for obtaining monomers for polymerization, shown in Examples 28 to 32, StartSYNTH manufactured by Milestone General K.K. was used to perform depolymerization in the following conditions.

A pressure-resistant container was charged with a stirrer and 30 g of the polyamide, the polyamide resin composition and the crude polyamide, and 37% hydrochloric acid and distilled water were added thereto so that 11% hydrochloric acid was achieved. The resulting mixture was stirred at 140° C. for 1 hour. The temperature rise time was fixed to 20 minutes in the temperature rise process to a predetermined temperature. After depolymerization was performed, the resultant was naturally cooled in an apparatus and, when the temperature was dropped to 80° C., the pressure-resistant container was taken out.

Furthermore, in Examples 28 to 32, the actual recovery percentages of adipic acid and hexamethylenediamine actually recovered after purification were calculated by the following method. The mass of a pure polyamide component in the polyamide, the polyamide resin composition and the crude polyamide charged was calculated; the theoretically maximum mass of recovery was determined under the assumption that 100% of the pure polyamide component was depolymerized to generated 100% of a diamine and a dicarboxylic acid with no occurrence of any secondary reaction after depolymerization and also that 100% of such diamine and dicarboxylic acid were recovered with no physical loss during purification, the theoretically maximum mass of recovery being regarded as 100%; and the rate of the mass of adipic acid and hexamethylenediamine actually recovered was determined as the actual recovery percentage (%) based the theoretically maximum mass of recovery.

### <Normal Heating>

A pressure-resistant container made of PTFE (microwave-sample decomposition container (temperature measurement type) manufactured by Toeisha Co., Ltd.) was charged with a stirrer and predetermined amounts of the polyamide, the polyamide resin composition and the crude polyamide, and 37% hydrochloric acid and distilled water were added thereto so that a desired percentage (% by mass) was achieved. The resulting mixture was stirred with an oil bath at a predetermined temperature for a desired time.

The depolymerization time was a time after the inner temperature reached a predetermined temperature, and the temperature rise time with each oil bath was shown in Table 4 and Table 10.

The depolymerization time was fixed to 1 hour and the depolymerization temperature was changed in microwave heating and normal heating. The results are shown in Table 4.

The depolymerization time and the depolymerization temperature were changed in microwave heating and normal heating. The results are shown in Table 5.

### <Measurement of Depolymerization Percentage>

### [Evaluation of Amount of Hexamethylenediamine (HMD) and its Derivative Relative to Polyamide Before Depolymerization Step and Evaluation of Amount of Adipic Acid (ADA) and its Derivative Relative to Polyamide Before Depolymerization Step]

The reaction liquid after the depolymerization step was sampled and subjected to measurement by a NMR method to calculate the depolymerization percentage (%) of the polyamide.

The solution after hydrolysis, obtained in each of Examples and Comparative Examples, was added to a NMR tube having a diameter of 5 mm, a special sample tube N-502B (Nihon Seimitsu Kagaku Co., Ltd.) for NMR, in which a measurement portion was filled with benzene d-6, was inserted into the above NMR tube having a diameter of 5 mm, and measurement was performed using a NMR apparatus (ECZ-500) manufactured by JEOL Ltd., in conditions of 1H as an observation nucleus, a measurement temperature of 25° C., and a number of scans of 1024.

The (Value of integral of hexamethylenediamine)/(Value of integral of hexamethylenediamine+Value of integral of amide bond) was calculated from the values of integral of hexamethylenediamine and an amide bond, and was used as the depolymerization percentage of the polyamide.

### [Separation Step]

The solution after the depolymerization step was warmed, and subjected to hot filtration at 85° C., to remove the solid content (including a glass fiber, a silicon coating, a metal piece, and the like) as an insoluble fraction.

Next, the filtrate was cooled to ordinary temperature to precipitate a dicarboxylic acid as a crystal. By filtration, the crystal of the dicarboxylic acid was separated from the filtrate.

### [Purification Step]

### (Purification of Dicarboxylic Acid)

The crystal of the dicarboxylic acid, obtained in the separation step, was dissolved in water by dissolution in an amount equal to or more than the mass of the crystal and warming to 80° C., and the resultant was left to still stand after the dissolution and thus cooled, to recrystallize the dicarboxylic acid. The precipitated crystal of the dicarboxylic acid was recovered by filtration.

### (Purification of Diamine)

Sodium hydroxide was gradually added to the filtrate obtained in the separation step such that the amount of sodium hydroxide added was an equal mole or more relative to that of hydrochloric acid, and it was confirmed that a salt was precipitated. The reaction liquid after neutralization was distilled with Kugelrohr. After heating at 100° C. and 300 mbar, the resultant was gradually heated to 110° C. and depressurized to 160 mbar, retained for 3 hours, further heated to 140° C. and depressurized to 80 mbar, and retained for about 1 hour, thereby recovering a finally objective diamine.

### (Analysis of Monomers)

The diamine and the dicarboxylic acid after purification were subjected to NMR measurement, and it was confirmed that they were objective substances.

A trace of impurities was analyzed by an ICP-AES semiquantitative method. The analyzer used here was an ICP emission spectrophotometer SPS3520UV-DD manufactured by Hitachi High-Tech Corporation (SII).

### [Method for Forming Polyamide by Polymerization]

A polyamide formation method by polymerization was carried out by “hot melt polymerization method”, as follows.

In 150 g of distilled water were dissolved 150 g of an equimolar salt of the adipic acid and the hexamethylenediamine recovered as described above, to produce an aqueous 50% by mass homogeneous solution of equimolar feedstock monomers. An autoclave having an inner volume of 0.5 L was charged with the aqueous solution, and purged with nitrogen.

The aqueous solution was concentrated by gradually evacuation water vapor with stirring at a temperature of 110 to 150° C. until a solution concentration of 70% by mass was achieved. Thereafter, the interior temperature was raised to 220° C. The pressure of the autoclave was here increased to 1.8 MPa. While water vapor was gradually evaluated as it was for 1 hour until the interior temperature reached 245° C. with the pressure being kept at 1.8 MPa, a reaction was allowed to occur for 1 hour.

Next, the pressure was dropped over 1 hour.

Thereafter, the content of the autoclave was kept under a reduced pressure of 650 torr (86.66 kPa) using a vacuum apparatus for 10 minutes. The final interior temperature in polymerization was 265° C.

Thereafter, a strand was formed through a lower spinning port (nozzle) by pressurization with nitrogen, cooled with water and cut, then discharged in the form of a pellet, and dried under a nitrogen atmosphere at 100° C. for 12 hours, to obtain a polyamide. The Mw was 35000 and the Mw/Mn was 2.0.

### (Analysis of Polyamide)

The resulting polyamide was subjected to NMR measurement and it was confirmed that it was polyamide 66.

A trace of impurities was analyzed by an ICP-AES semiquantitative method. The analyzer used here was an ICP emission spectrophotometer SPS3520UV-DD manufactured by Hitachi High-Tech Corporation (SII).

### [Evaluation of Corrosion Resistance]

The corrosion resistance was evaluated from an example of zirconium, which was associated with the graph between the hydrochloric acid concentration and the temperature described in “Properties of Tantalum for Applications in the Chemical Process Industry: F. J. Hunkeler (USA)ASTM STP849, 1984,P28-49”.

A case of corrosion exceeding a line of 5 mpy (5 mil per year) was graded as “X”, a case of corrosion around the line was graded as “Δ”, and cases of corrosion more apart, and much more apart, from the line were graded as “◯” and “⊚”, respectively.

TABLE 4

| | Comparative | Comparative | Comparative | Comparative | | | |
|---|---|---|---|---|---|---|---|
| | Example 1 | Example 2 | Example 3 | Example 4 | Example 1 | Example 2 | Example 3 |
| Sample liquid | Sample | Sample | Sample | Sample | Sample | Sample | Sample |
| | liquid 2 | liquid 2 | liquid 2 | liquid 2 | liquid 2 | liquid 2 | liquid 2 |
| Acid | Hydrochloric | Hydrochloric | Hydrochloric | Hydrochloric | Hydrochloric | Hydrochloric | Hydrochloric |
| | acid | acid | acid | acid | acid | acid | acid |
| Acid concentration | 17 | 17 | 17 | 17 | 17 | 17 | 17 |
| (% by mass) | | | | | | | |
| pKa | −8.0 | −8.0 | −8.0 | −8.0 | −8.0 | −8.0 | −8.0 |
| Polyamide | Leona 1300 | Leona 1300 | Leona 1300 | Leona 1300 | Leona 1300 | Leona 1300 | Leona 1300 |
| Heating method | Microwave | Oil bath | Microwave | Microwave | Microwave | Microwave | Oil bath |
| Temperature rise | 0.27 | 0.18 | 0.27 | 0.27 | 0.27 | 0.27 | 0.55 |
| time (h) | | | | | | | |
| Depolymerization | 1 | 1 | 1 | 1 | 1 | 1 | 1 |
| time (h) | | | | | | | |
| Depolymerization | 100 | 100 | 105 | 110 | 115 | 120 | 120 |
| temperature (° C) | | | | | | | |
| Depolymerization | 37 | 29 | 48 | 62 | 80 | 88 | 86 |
| percentage (%) | | | | | | | |
| Corrosion | ⊚ | ⊚ | ⊚ | ⊚ | ⊚ | ⊚ | ⊚ |
| resistance | | | | | | | |

| | Example 4 | Example 5 | Example 6 | Example 7 | Example 8 |
|---|---|---|---|---|---|
| Sample liquid | Sample | Sample | Sample | Sample | Sample |
| | liquid 2 | liquid 2 | liquid 2 | liquid 2 | liquid 2 |
| Acid | Hydrochloric | Hydrochloric | Hydrochloric | Hydrochloric | Hydrochloric |
| | acid | acid | acid | acid | acid |
| Acid concentration | 17 | 17 | 17 | 17 | 17 |
| (% by mass) | | | | | |
| pKa | −8.0 | −8.0 | −8.0 | −8.0 | −8.0 |
| Polyamide | Leona 1300 | Leona 1300 | Leona 1300 | Leona 1300 | Leona 1300 |
| Heating method | Microwave | Microwave | Oil bath | Microwave | Oil bath |
| Temperature rise | 0.27 | 0.27 | 0.55 | 0.27 | 0.58 |
| time (h) | | | | | |
| Depolymerization | 1 | 1 | 1 | 1 | 1 |
| time (h) | | | | | |
| Depolymerization | 130 | 140 | 140 | 160 | 160 |
| temperature (° C) | | | | | |
| Depolymerization | 98 | 100 | 100 | 100 | 100 |
| percentage (%) | | | | | |
| Corrosion | ⊚ | ⊚ | ⊚ | ◯ | ◯ |
| resistance | | | | | |

TABLE 5

| | Comparative | Comparative | Comparative | Comparative | Comparative |
|---|---|---|---|---|---|
| | Example 1 | Example 5 | Example 6 | Example 4 | Example 7 |
| Sample liquid | Sample | Sample | Sample | Sample | Sample |
| | liquid 2 | liquid 2 | liquid 2 | liquid 2 | liquid 2 |
| Acid | Hydrochloric | Hydrochloric | Hydrochloric | Hydrochloric | Hydrochloric |
| | acid | acid | acid | acid | acid |
| Acid concentration | 17 | 17 | 17 | 17 | 17 |
| (% by mass) | | | | | |
| pKa | −8.0 | −8.0 | −8.0 | −8.0 | −8.0 |
| Polyamide | Leona 1300 | Leona 1300 | Leona 1300 | Leona 1300 | Leona 1300 |
| Heating method | Microwave | Microwave | Microwave | Microwave | Microwave |
| Depolymerization | 1 | 2 | 3 | 1 | 2 |
| time (h) | | | | | |
| Depolymerization | 100 | 100 | 100 | 110 | 110 |
| temperature (° C.) | | | | | |
| Depolymerization | 37 | 52 | 70 | 62 | 75 |
| percentage (%) | | | | | |
| Corrosion | ⊚ | ⊚ | ⊚ | ⊚ | ⊚ |
| resistance | | | | | |
| | Example 9 | Example 10 | Example 11 | Example 12 | Example 13 |
| Sample liquid | Sample | Sample | Sample | Sample | Sample |
| | liquid 2 | liquid 2 | liquid 2 | liquid 2 | liquid 2 |
| Acid | Hydrochloric | Hydrochloric | Hydrochloric | Hydrochloric | Hydrochloric |
| | acid | acid | acid | acid | acid |
| Acid concentration | 17 | 17 | 17 | 17 | 17 |
| (% by mass) | | | | | |
| pKa | −8.0 | −8.0 | −8.0 | −8.0 | −8.0 |
| Polyamide | Leona 1300 | Leona 1300 | Leona 1300 | Leona 1300 | Leona 1300 |
| Heating method | Microwave | Microwave | Microwave | Microwave | Microwave |
| Depolymerization | 4 | 6 | 2.5 | 3 | 4 |
| time (h) | | | | | |
| Depolymerization | 100 | 100 | 110 | 110 | 110 |
| temperature (° C.) | | | | | |
| Depolymerization | 80 | 91 | 80 | 86 | 92 |
| percentage (%) | | | | | |
| Corrosion | ⊚ | ⊚ | ⊚ | ⊚ | ⊚ |
| resistance | | | | | |
| | Example 14 | Example 2 | Example 15 | Example 16 | Example 17 |
| Sample liquid | Sample | Sample | Sample | Sample | Sample |
| | liquid 2 | liquid 2 | liquid 2 | liquid 2 | liquid 2 |
| Acid | Hydrochloric | Hydrochloric | Hydrochloric | Hydrochloric | Hydrochloric |
| | acid | acid | acid | acid | acid |
| Acid concentration | 17 | 17 | 17 | 17 | 17 |
| (% by mass) | | | | | |
| pKa | −8.0 | −8.0 | −8.0 | −8.0 | −8.0 |
| Polyamide | Leona 1300 | Leona 1300 | Leona 1300 | Leona 1300 | Leona 1300 |
| Heating method | Microwave | Microwave | Microwave | Microwave | Microwave |
| Depolymerization | 6 | 1 | 2 | 3 | 0.5 |
| time (h) | | | | | |
| Depolymerization | 110 | 120 | 120 | 120 | 160 |
| temperature (° C.) | | | | | |
| Depolymerization | 98 | 88 | 97 | 100 | 100 |
| percentage (%) | | | | | |
| Corrosion | ⊚ | ⊚ | ⊚ | ⊚ | ◯ |
| resistance | | | | | |

TABLE 6

| | Comparative | Comparative | | | |
|---|---|---|---|---|---|
| | Example 8 | Example 9 | Example 18 | Example 19 | Example 20 |
| Sample liquid | Sample liquid 7 | Sample liquid 6 | Sample liquid 5 | Sample liquid 8 | Sample liquid 9 |
| Acid | Adipic acid | Phosphoric acid | Sulfuric acid | Methanesulfonic | p-Toluenesulfonic |
| | | | | acid | acid |
| Acid concentration (% by mass) | 30 | 21 | 30 | 23 | 36 |
| pKa | 4.42 | 2.12 | −3.0 | −2.6 | −2.8 |
| Polyamide | Leona 1300 | Leona 1300 | Leona 1300 | Leona 1300 | Leona 1300 |
| Heating method | Microwave | Microwave | Microwave | Microwave | Microwave |
| Depolymerization time (h) | 1 | 1 | 1 | 1 | 1 |
| Depolymerization temperature (° C.) | 140 | 140 | 140 | 140 | 140 |
| Depolymerization percentage (%) | 0 | — (Not | 100 | 100 | 100 |
| | | dissolved) | | | |
| Corrosion resistance | ⊚ | ⊚ | ⊚ | ⊚ | ⊚ |

TABLE 7

| | Comparative | Comparative | Comparative | Comparative | Comparative | Comparative |
|---|---|---|---|---|---|---|
| | Example 10 | Example 11 | Example 12 | Example 1 | Example 3 | Example 4 |
| Sample liquid | Sample | Sample | Sample | Sample | Sample | Sample |
| | liquid 1 | liquid 1 | liquid 1 | liquid 2 | liquid 2 | liquid 2 |
| Acid | Hydrochloric | Hydrochloric | Hydrochloric | Hydrochloric | Hydrochloric | Hydrochloric |
| | acid | acid | acid | acid | acid | acid |
| Acid concentration | 35 | 35 | 35 | 17 | 17 | 17 |
| (% by mass) | | | | | | |
| pKa | −8.0 | −8.0 | −8.0 | −8.0 | −8.0 | −8.0 |
| Polyamide | Leona 1300 | Leona 1300 | Leona 1300 | Leona 1300 | Leona 1300 | Leona 1300 |
| Heating method | Microwave | Microwave | Microwave | Microwave | Microwave | Microwave |
| Depolymerization | 1 | 1 | 1 | 1 | 1 | 1 |
| time (h) | | | | | | |
| Depolymerization | 100 | 120 | 130 | 100 | 105 | 110 |
| temperature (° C.) | | | | | | |
| Depolymerization | 18 | 56 | 72 | 37 | 48 | 62 |
| percentage (%) | | | | | | |
| Corrosion | ◯ | ◯ | Δ | ⊚ | ⊚ | ⊚ |
| resistance | | | | | | |

| | Comparative | Comparative | Comparative | Comparative |
|---|---|---|---|---|
| | Example 13 | Example 14 | Example 15 | Example 16 |
| Sample liquid | Sample | Sample | Sample | Sample |
| | liquid 3 | liquid 3 | liquid 4 | liquid 4 |
| Acid | Hydrochloric | Hydrochloric | Hydrochloric | Hydrochloric |
| | acid | acid | acid | acid |
| Acid concentration | 14 | 14 | 12 | 12 |
| (% by mass) | | | | |
| pKa | −8.0 | −8.0 | −8.0 | −8.0 |
| Polyamide | Leona 1300 | Leona 1300 | Leona 1300 | Leona 1300 |
| Heating method | Microwave | Microwave | Microwave | Microwave |
| Depolymerization | 1 | 1 | 1 | 1 |
| time (h) | | | | |
| Depolymerization | 100 | 110 | 100 | 115 |
| temperature (° C.) | | | | |
| Depolymerization | 36 | 60 | 37 | 66 |
| percentage (%) | | | | |
| Corrosion | ⊚ | ⊚ | ⊚ | ⊚ |
| resistance | | | | |

TABLE 8

| | Example 21 | Example 22 | Example 1 | Example 2 | Example 4 | Example 5 |
|---|---|---|---|---|---|---|
| Sample liquid | Sample | Sample | Sample | Sample | Sample | Sample |
| | liquid 1 | liquid 1 | liquid 2 | liquid 2 | liquid 2 | liquid 2 |
| Acid | Hydrochloric | Hydrochloric | Hydrochloric | Hydrochloric | Hydrochloric | Hydrochloric |
| | acid | acid | acid | acid | acid | acid |
| Acid concentration | 35 | 35 | 17 | 17 | 17 | 17 |
| (% by mass) | | | | | | |
| pKa | −8.0 | −8.0 | −8.0 | −8.0 | −8.0 | −8.0 |
| Polyamide | Leona 1300 | Leona 1300 | Leona 1300 | Leona 1300 | Leona 1300 | Leona 1300 |
| Heating method | Microwave | Microwave | Microwave | Microwave | Microwave | Microwave |
| Depolymerization | 1 | 1 | 1 | 1 | 1 | 1 |
| time (h) | | | | | | |
| Depolymerization | 140 | 160 | 115 | 120 | 130 | 140 |
| temperature (° C.) | | | | | | |
| Depolymerization | 94 | 100 | 80 | 88 | 98 | 100 |
| percentage (%) | | | | | | |
| Corrosion resistance | Δ | Δ | ⊚ | ⊚ | ⊚ | ⊚ |

| | Example 23 | Example 24 | Example 25 | Example 26 | Example 27 |
|---|---|---|---|---|---|
| Sample liquid | Sample | Sample | Sample | Sample | Sample |
| | liquid 3 | liquid 3 | liquid 4 | liquid 4 | liquid 4 |
| Acid | Hydrochloric | Hydrochloric | Hydrochloric | \|Hydrochloric | Hydrochloric |
| | acid | acid | acid | acid | acid |
| Acid concentration | 14 | 14 | 12 | 12 | 12 |
| (% by mass) | | | | | |
| pKa | −8.0 | −8.0 | −8.0 | −8.0 | −8.0 |
| Polyamide | Leona 1300 | Leona 1300 | Leona 1300 | Leona 1300 | Leona 1300 |
| Heating method | Microwave | Microwave | Microwave | Microwave | Microwave |
| Depolymerization | 1 | 1 | 1 | 1 | 1 |
| time (h) | | | | | |
| Depolymerization | 120 | 140 | 120 | 130 | 140 |
| temperature (° C.) | | | | | |
| Depolymerization | 90 | 100 | 88 | 98 | 100 |
| percentage (%) | | | | | |
| Corrosion resistance | ⊚ | ⊚ | ⊚ | ⊚ | ⊚ |

TABLE 9

| | Example 28 | Example 29 | Example 30 | Example 31 | Example 32 |
|---|---|---|---|---|---|
| Sample liquid | Sample liquid 2 | Sample liquid 10 | Sample liquid 11 | Sample liquid 12 | Sample liquid 13 |
| Acid | Hydrochloric acid | Hydrochloric acid | Hydrochloric acid | Hydrochloric acid | Hydrochloric acid |
| Acid concentration (% by mass) | 17 | 17 | 17 | 17 | 17 |
| pKa | −8.0 | −8.0 | −8.0 | −8.0 | −8.0 |
| Polyamide | Leona 1300 | Leona 14G33 | Radiator tank | Air-bag (with Si) | Air-bag (without Si) |
| Heating method | Microwave | Microwave | Microwave | Microwave | Microwave |
| Depolymerization time (h) | 1 | 1 | 1 | 1 | 1 |
| Depolymerization temperature (° C.) | 140 | 140 | 140 | 140 | 140 |
| Depolymerization percentage (%) | 100 | 100 | 100 | 100 | 100 |
| Purity of ADA (%) | >99 | >99 | >99 | >99 | >99 |
| Content of Si (ppm) | <5.0 | 110 | 54 | 100 | 89 |
| Actual recovery rate of ADA (%) | 86 | 84 | 82 | 85 | 86 |
| Purity of HMD (%) | >99 | >99 | >99 | >99 | >99 |
| Content of Si (ppm) | <5.0 | <5.0 | <5.0 | <5.0 | <5.0 |
| Actual recovery rate of HMD (%) | 89 | 88 | 87 | 88 | 89 |
| Polyamide polymerization | Polymerizable | Polymerizable | Polymerizable | Polymerizable | Polymerizable |
| Content of Si (ppm) | <5.0 | 50 | 30 | 45 | 35 |

TABLE 10

| | Example 5 | Example 33 | Example 34 | Example 35 | Example 36 | Example 37 |
|---|---|---|---|---|---|---|
| Sample liquid | Sample | Sample | Sample | Sample | Sample | Sample |
| | liquid 2 | liquid 14 | liquid 15 | liquid 16 | liquid 17 | liquid 18 |
| Acid | Hydrochloric | Hydrochloric | Hydrochloric | Hydrochloric | Hydrochloric | Hydrochloric |
| | acid | acid | acid | acid | acid | acid |
| Acid concentration | 17 | 17 | 17 | 17 | 17 | 17 |
| (% by mass) | | | | | | |
| pKa | −8.0 | −8.0 | −8.0 | −8.0 | −8.0 | −8.0 |
| Polyamide | Leona 1300 | Leona 1300 | Leona 1300 | Leona 1300 | Leona 1300 | Leona 1300 |
| Amount of polyamide (g) | 1.12 | 2.5 | 3 | 3.5 | 4 | 4.5 |
| Proton of hydrochloric acid/ | 5.32 | 2.38 | 1.99 | 1.7 | 1.49 | 1.32 |
| Amide group (molar ratio) | | | | | | |
| Heating method | Microwave | Oil bath | Microwave | Microwave | Microwave | Microwave |
| Temperature rise time (h) | 0.27 | 0.27 | 0.27 | 0.27 | 0.27 | 0.27 |
| Depolymerization time (h) | 1 | 1 | 1 | 1 | 1 | 1 |
| Depolymerization | 140 | 140 | 140 | 140 | 140 | 140 |
| temperature (° C.) | | | | | | |
| Depolymerization | 100 | 100 | 100 | 99 | 99 | 98 |
| percentage (%) | | | | | | |
| Corrosion resistance | ⊚ | ⊚ | ⊚ | ⊚ | ⊚ | ⊚ |

In each Example, depolymerization was performed at a depolymerization percentage of 80% or more to obtain the effect, a high efficiency in actual monomer recovery through separation/purification. Specifically, the actual monomer recovery efficiency was 82% or more as shown in Examples 28 to 32. In Comparative Examples, the actual monomer recovery efficiency was 75% even in Comparative Example 7 where the depolymerization percentage was highest. If all of 75% of the decomposed fraction was changed to adipic acid and hexamethylenediamine with no secondary reaction at all, the entire amount of which was successfully recovered with no physical loss during purification, the actual recovery percentage did not exceed 75%, and therefore it was considered that the recovery percentage in Comparative Example 7 did not exceed 82%, which was the lowest actual recovery percentage in Examples.

In each Example, it was confirmed that the degree of corrosion in a reaction apparatus was low and that hexamethylenediamine and adipic acid were obtained at high yields and high purities from various polyamide-containing waste materials, and it was also confirmed that polyamide 66 was obtained by polymerization by using hexamethylenediamine and adipic acid after purification.

The present application is based on the Japanese Patent Application (Japanese Patent Application No. 2023-165285) filed with JPO on Sep. 27, 2023, the content of which is herein incorporated as reference.

### INDUSTRIAL APPLICABILITY

The method for producing a diamine and a dicarboxylic acid, and the method for recycling a polyamide of the present invention have industrial applicability in an efficient recycling method of a polyamide resin, a polyamide fiber, a polyamide resin composition, and a molded product used for automobile parts or various industrial parts.

1. A method for producing a diamine and a dicarboxylic acid, the method comprising:
pre-treatment by subjecting a polyamide-containing waste material to one or more selected from the group consisting of pulverization, washing, and foreign substance segregation, to obtain a crude polyamide,
depolymerization by adding the crude polyamide to an acid-containing aqueous solution in a tightly closed reactor, raising a temperature to a target temperature of 90° C. or more and 160° C. or less, and then performing heating at or around the target temperature, to depolymerize 80% or more of an amide group in the entire polyamide contained in the crude polyamide to decompose the polyamide into a diamine and a dicarboxylic acid,
separation by removing a component other than the diamine and a derivative of the diamine as well as the dicarboxylic acid and a derivative of the dicarboxylic acid, from a reaction liquid obtained in the depolymerization, to obtain the diamine and the derivative of the diamine as well as the dicarboxylic acid and the derivative of the dicarboxylic acid, and
purification by isolating and purifying each of the diamine and the dicarboxylic acid, from the diamine and the derivative of the diamine as well as the dicarboxylic acid and the derivative of the dicarboxylic acid obtained in the separation.

2. The method for producing the diamine and the dicarboxylic acid according to claim 1, wherein, in a state where a space not filled with an aqueous solution containing the crude polyamide is present on an upper side in the reactor, and a microwave is radiated from the upper side in the reactor using a microwave transmitter through a waveguide and the space unfilled, in the depolymerization.

3. The method for producing the diamine and the dicarboxylic acid according to claim 1, wherein a rate of temperature rise in the temperature rise is 25° C./min or less in the depolymerization.

4. The method for producing the diamine and the dicarboxylic acid according to claim 1, wherein the reactor comprises equipment which monitors a pressure in the reactor and performs evacuation when the pressure exceeds a reference pressure, in the depolymerization.

5. The method for producing the diamine and the dicarboxylic acid according to claim 1, wherein the separation comprises removing, as an insoluble fraction, a component other than the diamine and the derivative of the diamine as well as the dicarboxylic acid and the derivative of the dicarboxylic acid from the reaction liquid, in a state where a portion or an entire amount of the dicarboxylic acid and/or the derivative of the dicarboxylic acid and the diamine and the derivative of the diamine is dissolved in the reaction liquid.

6. The method for producing the diamine and the dicarboxylic acid according to claim 1, wherein a pKa of the acid is 0 or less.

7. The method for producing the diamine and the dicarboxylic acid according to claim 1, wherein an amount of the acid in terms of a molar ratio of an amide group of the crude polyamide and a proton of the acid satisfies the following: Amide group of crude polyamide:Proton of acid=1:1 to 1:5.5.

8. The method for producing the diamine and the dicarboxylic acid according to claim 1, wherein the acid is hydrochloric acid.

9. The method for producing the diamine and the dicarboxylic acid according to claim 8, wherein a concentration of the hydrochloric acid in the aqueous solution is 5% by mass or more and 25% by mass or less.

10. The method for producing the diamine and the dicarboxylic acid according to claim 1, wherein a main component of a polyamide in the polyamide-containing waste material is polyamide 66.

11. The method for producing the diamine and the dicarboxylic acid according to claim 1, wherein the reactor used in the depolymerization has an inner surface made of a material including a glass lining, zirconium, or tantalum.

12. The method for producing the diamine and the dicarboxylic acid according to claim 1, wherein hot filtration and centrifugation are performed when the component other than the diamine and the derivative of the diamine as well as the dicarboxylic acid and the derivative of the dicarboxylic acid is removed in the separation.

13. The method for producing the diamine and the dicarboxylic acid according to claim 1, wherein in the purification, purification of the dicarboxylic acid is performed by crystallization.

14. The method for producing the diamine and the dicarboxylic acid according to claim 1, wherein in the purification, purification of the diamine is performed by distillation.

15. The method for producing the diamine and the dicarboxylic acid according to claim 2, wherein a partition window which separates the reactor and the microwave transmitter is present in the middle of the waveguide.

16. The method for producing the diamine and the dicarboxylic acid according to claim 15, wherein the partition window includes quartz glass.

17. The method for producing the diamine and the dicarboxylic acid according to claim 2, wherein a frequency of the microwave in heating by the microwave is 0.8 to 6 GHz.

18. The method for producing the diamine and the dicarboxylic acid according to claim 1, wherein in the purification, a content of a Si element reaches 1 ppm by mass or more and 200 ppm by mass or less based on a total amount of the dicarboxylic acid.

19. A method for recycling a polyamide, the method comprising:
polymerization by polymerizing a diamine and a dicarboxylic acid to obtain a polyamide, wherein the diamine and the dicarboxylic acid are obtained by the method for producing the diamine and the dicarboxylic acid according to claim 1.

20. A polyamide obtained by the method for recycling a polyamide according to claim 19, comprising
1 ppm by mass or more and 100 ppm by mass or less of a Si element based on a total amount of the polyamide.
